(19) United States
(12) Patent Application Publication
(10) Pub. No.: US 2024/0197912 A1
(43) Pub. Date: Jun. 20, 2024

(54) A PHARMACEUTICALLY ACCEPTABLE AQUEOUS GEL COMPOSITION FOR MRNA DELIVERY

(71) Applicant: MAGLE CHEMOSWED AB, Malmö (SE)

(72) Inventor: Håkan NYGREN, Billdal (SE)

(21) Appl. No.: 18/565,162

(22) PCT Filed: Jun. 3, 2022
(86) PCT No.: PCT/SE2022/050542

§ 371 Date: Nov. 29, 2023

(30) Foreign Application Priority Data
Jun. 3, 2021 (EP) 21177521.8

(51) Int. Cl. A61K 48/00; A61K 9/06; A61K 38/17; A61K 38/18; A61K 38/22; A61K 38/39; A61K 38/45; A61K 38/48; A61K 47/52; A61K 47/61; A61K 47/64

(52) U.S. Cl. CPC A61K 48/0041; A61K 9/06; A61K 38/1716; A61K 38/177; A61K 38/1777; A61K 38/18; A61K 38/1875; A61K 38/2271; A61K 38/39; A61K 38/45; A61K 38/482; A61K 38/4886; A61K 47/52; A61K 47/61; A61K 47/6435

(57) ABSTRACT

A pharmaceutically acceptable aqueous gel composition comprising a gelling agent, magnesium and/or manganese, or bivalent ions thereof, and an mRNA molecule encoding a protein of interest; and methods for inducing or facilitating repair, re-generation or generation of tissue in a human or animal subject, comprising administering the composition to the site of the tissue to be repaired, re-generated or generated.

### TECHNICAL FIELD

The present disclosure relates to a pharmaceutical compositions for delivery of mRNA, in particular pharmaceutically acceptable aqueous gel compositions and to methods for targeted delivery of mRNA to specific sites in situ.

### BACKGROUND ART

The first suggestion to use mRNA as a platform for vaccine development was made in 1989 (Malone et al., 1989). Later publications have presented different methods to enhance the uptake of external mRNA into cells, such as Ca and lipid-based nanoparticles (Schlake et al., 2012). In 2020 the first vaccines were produced by Pfizer/Biontech, followed by other pharmaceutical companies.

Compositions comprising osteotropic genes and a bone-compatible matrix such as a hydroxyapatite matrix for stimulation of bone growth has been suggested in e.g. WO9522611.

It has also been suggested to deliver RNA molecules of generally much shorter length as compared to mRNA, i.e. siRNA, to mouse embryonic stem cells through hydroxyapatite-based nanovehicles (Zantye et al. Mol. Pharmaceutics 2021, 18, 796-806).

Protocols for synthesis of biodegradable hydroxyapatite particles for drug and siRNA delivery has also been published (Liu et al., Journal of Colloid and Interface Science, 570 (2020) 402-410).

### SUMMARY

The present invention is based on the idea to administer a composition comprising mRNA encoding a protein of interest together with catalyzers of calcium apatite formation to induce the formation of mRNA-containing hydroxyapatite nanoparticles for delivery of mRNA to cells in situ. Such delivery is useful whenever it is desirable to express new proteins, modified proteins, or increased amounts of already expressed proteins. This may be desirable, e.g. in modification of cell function.

The present invention utilize the property of metals and metal oxides to catalyze the formation of calcium-phosphates like hydroxyapatite with mRNA added as a phosphate source, resulting in formation of mRNA-hydroxyapatite nanoparticles that are taken up by target cells. The mRNA molecules are protected from being decomposed by serum enzymes by being an integral part of such particles, and it also promotes the uptake of the mRNA together with the uptake of hydroxyapatite by the scavenger receptors of a target cell.

It has been found that providing metallic magnesium and/or manganese, or bivalent ions thereof, in an aqueous gel composition also comprising mRNA molecules induces the formation in situ of hydroxyapatite particles incorporating mRNA molecules. The hydroxyapatite particles are then readily absorbed by cells and subsequently degraded inside the cells to release the mRNA molecules, which is translated to corresponding proteins.

Thus, according to a first aspect of the invention, there is provided a pharmaceutically acceptable aqueous gel composition comprising a gelling agent, magnesium and/or manganese, or bivalent ions thereof, and an mRNA molecule encoding a protein of interest.

According to some embodiments, the magnesium and/or manganese, or bivalent ions thereof, is present in an amount sufficient to induce hydroxyapatite formation under physiological conditions.

According to some embodiments, the gelling agent is selected from the group consisting of starch or derivatives thereof; agar/agarose or derivatives of agar/agarose; hyaluronic acid or derivatives of hyaluronic acid; chitosan; gelatin; and dextran. In some embodiments, the concentration of the gelling agent is in the range 0.5-10 percent w/w.

According to some embodiments, the protein of interest is a protein involved in tissue repair, tissue re-generation, or tissue generation.

According to some embodiments, the protein is selected from the group consisting of Phosphate-regulating neutral endopeptidase, X-linked (encoded by PHEX), heat shock protein 90-alpha (encoded by HSP90AA1), Chordin Like 2 (encoded by CHRDL2), short transient receptor potential channel 4 (encoded by TRPC4), pannexin 3 (encoded by PANX3), Collagen Type XXIV Alpha 1 (encoded by COL24A1), the gene product of ATP283, Pleckstrin Homology Domain Containing B1 (encoded by PLEKHB1), Leukocyte immunoglobulin-like receptor subfamily B member 4 (encoded by LILRB4), Anoctamin 5 (encoded by ANO5), ChaC Glutathione Specific Gamma-Glutamylcyclotransferase 1 (encoded by CHAC1), Dynein Axonemal Intermediate Chain 2 (encoded by DNAI2), Aggrecan (encoded by ACAN), Integrin Subunit Alpha 10 (encoded by ITGA10), Fibromodulin (encoded by FMOD), Secreted Phosphoprotein 1 (a.k.a. osteopontin, encoded by SPP1), Apolipoprotein C1 (encoded by APOC1), Patched 2 (encoded by PTCH2), Apolipoprotein E (encoded by APOE), Triggering Receptor Expressed On Myeloid Cells 2 (encoded by TREM2), podoplanin (encoded by PDPN), Solute Carrier Family 13 Member 5 (encoded by SLC13A5), Inducible T Cell Costimulator (encoded by ICOS), Formyl Peptide Receptor 2 (encoded by FPR2), Matrix Metallopeptidase 12 (encoded by MMP12), Tenascin N (encoded by TNN), Asporin (encoded by ASPN), Granzyme A (encoded by GZMA), Osteoglycin (encoded by OGN), Wnt Family Member 2 (encoded by WNT2), Neuropeptide Y (encoded by NPY), Bone Morphogenetic Protein 2 (encoded by BMP-2), Family With Sequence Similarity 169 Member A (encoded by FAM169), Retinol Binding Protein 1 (encoded by RBP1), ISG15 Ubiquitin Like Modifier (encoded by ISG15), Tribbles Pseudokinase 3 (encoded by TRIB3), Ubiquitin Specific Peptidase 18 (encoded by USP18), Claudin 1 (encoded by CLDN1), Myocilin (encoded by MYOC), C-X-C Motif Chemokine Ligand 9 (encoded by CXCL9), Interferon Induced Protein With Tetratricopeptide Repeats 3 (encoded by IFIT3), RRAD, Ras Related Glycolysis Inhibitor And Calcium Channel Regulator (encoded by RRAD) Interferon Alpha Inducible Protein 27 (encoded by/F127).

According to one aspect, the present invention relates to the above composition for use in a method for inducing or facilitating repair, re-generation or generation of tissue in a human or animal subject, said method the composition comprises administering said composition to the site of the tissue to be repaired, re-generated or generated.

According to some embodiments, the tissue is bone tissue.

In some embodiments, such methods are performed in connection with spinal fusion, and implantation of implants in bone tissue, such as prosthetic joints and dental implants.

In some embodiments, such methods are performed to facilitate or improve healing of tissue damaged due to trauma and/or disease, such as fractures and traumatic bone rift injuries.

In one aspect, the invention relates to a method for inducing or facilitating repair, re-generation or generation of tissue in a human or animal subject, said method comprising administering a composition according to the invention, to the site of the tissue to be repaired, re-generated or generated.

In one embodiment, the tissue is bone tissue.

In some embodiments, such methods are performed in connection with spinal fusion, and implantation of implants in bone tissue, such as prosthetic joints and dental implants.

In some embodiments, such methods are performed to facilitate or improve healing of tissue damaged due to trauma and/or disease, such as fractures and traumatic bone rift injuries.

The subject to be treated with the composition and methods according to the invention may be a human or animal subject. Animal subjects include mammalian species, such as horse, cat, dog, cow, pig, sheep, camel, and rodents including mouse and rat. When preparing a composition according to the invention for use in a non-human species, the mRNA sequence encoding the protein of interest may be a sequence encoding the protein of interest from the relevant species.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps. Furthermore, all published documents cited herein are incorporated by reference in their entirety.

### BRIEF DESCRIPTIONS OF THE DRAWINGS

FIG. 1 is a diagram showing the relative intensity (number of ions detected, normalised to the total ion detection) of Ca₃PO₅⁺ ions detected by tof-sims during analysis of MnO preincubated in DMEM (exposed) or saline (control).

FIG. 2: Human embryonic stem cells (SA16.7MFG-hesc) grown for 24 h exposed to 0.5 mg/ml of MnO preincubated in DMEM. Von Kossa staining reveals precipitate of HA within cells and in the extracellular space. The surface coverage of adhered cells is >80% within the colonies.

FIG. 3: Detection of secondary ions, specific for nitrogen bases, by ToF-SIMS after incubation of MnO or MgO with RNA in either cell culture medium (DMEM) or saline control.

FIG. 4: Detection of secondary ions, specific for nitrogen bases, by ToF-SIMS after incubation of MnO or MgO with RNA in cell culture medium (DMEM) containing three different chemically modified starch gels.

### DETAILED DESCRIPTION

Calcium phosphates, including hydroxyapatite (HA), is formed on the surface of corroding Mg²⁺ (Nygren et al., 2017) and Mn²⁺ (cf. Example 1) under physiological conditions.

It is known that nucleic acid molecules, such as RNA, bind to hydroxyapatite particles in phosphate buffered solution (Fadrosh et al., 2011). Thus, when mRNA is available as a phosphate source in the presence of metal Mg or Mn, or bivalent ions thereof, under physiological conditions, mRNA molecules will be incorporated into hydroxyapatite on Mg- or Mn-particles (cf. Example 2).

That RNA present as an integral part of hydroxyapatite particles are resistant to degradation by serum enzymes and are readily taken up by target cells has been shown in the context of small interfering RNA (siRNA) (Zantye et al., 2021). That HA-siRNA particles are dissolved in the cytoplasm of cells and release siRNA has also been shown (Liu et al., 2020). When applied to mRNA, the released mRNA molecules will be translated by the target cell into the protein of interest (cf. Example 3).

The first aspect of the present disclosure shows a pharmaceutically acceptable aqueous gel composition comprising a gelling agent, magnesium and/or manganese, or bivalent ions thereof, and an mRNA molecule encoding a protein of interest.

In one embodiment, the magnesium and/or manganese, or bivalent ions thereof, is present in an amount sufficient to induce hydroxyapatite formation under physiological conditions, such as on incubation in a cell culture medium or in situ when administered to a subject.

The function of the gel is to keep the mRNA and metal ions at, or in close proximity to, the site of injection and minimize leakage into the surrounding tissue. The gel should be pharmaceutically acceptable for drug delivery, and biodegradable, to allow release of the mRNA from the mRNA-containing hydroxyapatite particles at the site of application in the tissue. The type of gel that can be used is exemplified by, but not restricted to, agar/agarose based gels or derivatives of agar/agarose, gels based on hyaluronic acid or derivatives of hyaluronic acid, gels based on chitosan, gelatin, dextran or starch or derivatives thereof. The concentration of the gel should be in the range of 0.5 through 10 percent w/w. Guidance on preparation of pharmaceutically acceptable gel formulations may be found e.g. in “Pharmaceutics: The Science of Dosage Forms”, (Aulton, 2002); “Encyclopedia of Pharmaceutical Technology”, (Swarbrick, 2006); “Modern Pharmaceutics”, (Banker et al., 2002) “The Theory and Practice of Industrial Pharmacy”, (Lachman et al., 1986), all incorporated herein by reference.

In a preferred embodiment, the gelling agent is starch. Starch is well-known for use in pharmaceutical compositions and are available from a number of sources and commercial suppliers. Starch as a pharmaceutical excipient is generally produced from maize, potato, tapioca or rice, and is further described e.g. in the European Pharmacopoeia. Compositions comprising Degradable Starch Microspheres (DSM) have been used for drug delivery and other applications in medicine, such as detection of body-fluid leakage (e.g. WO2019/122120) and transarterial chemoembolization (Ludwig et al., 2021) and are useful in the present invention. Methods for producing DSM is i.a. disclosed in U.S. Pat. No. 4,124,705. The starch may be cross-linked, as has been described in the art (Atyabi, et al., 2006), (Fang, et al., 2008). Exemplary cross-linking agents are glutaraldehyde, formaldehyde, epichlorohydrine, and sodium trimetaphosphate.

In some embodiments, the aqueous gel composition according to the invention is acidic, i.e. has a pH<7, such as a pH of below 6.5, 6.0, 5.5, 5.0, 4.5, or 4.0. In some embodiments, the aqueous gel composition according to the invention is basic, i.e. has a pH>7, such as a pH above 7.5, 8.0, or 8.5. It is generally regarded that a pharmaceutically acceptable solution has a pH between about 4.5 and about 8.0.

In some embodiments, the protein of interest is a protein of human origin, encoded by a human gene.

The expression of mRNA in healing rat tibia was analysed as described by Uhlen et al. (Uhlén et al., 2015). Genes expressed in healing bone but not in untreated controls include the genes encoding proteins selected from the group consisting of Phosphate-regulating neutral endopeptidase, X-linked (encoded by PHEX), heat shock protein 90-alpha (encoded by HSP90AA1), Chordin Like 2 (encoded by CHRDL2), short transient receptor potential channel 4 (encoded by TRPC4), pannexin 3 (encoded by PANX3), Collagen Type XXIV Alpha 1 (encoded by COL24A1), the gene product of ATP283, Pleckstrin Homology Domain Containing B1 (encoded by PLEKHB1), Leukocyte immunoglobulin-like receptor subfamily B member 4 (encoded by LILRB4), Anoctamin 5 (encoded by ANO5), ChaC Glutathione Specific Gamma-Glutamylcyclotransferase 1 (encoded by CHAC1), Dynein Axonemal Intermediate Chain 2 (encoded by DNAI2), Aggrecan (encoded by ACAN), Integrin Subunit Alpha 10 (encoded by ITGA10), Fibromodulin (encoded by FMOD), Secreted Phosphoprotein 1 (a.k.a. osteopontin, encoded by SPP1), Apolipoprotein C1 (encoded by APOC1), Patched 2 (encoded by PTCH2), Apolipoprotein E (encoded by APOE), Triggering Receptor Expressed On Myeloid Cells 2 (encoded by TREM2), podoplanin (encoded by PDPN), Solute Carrier Family 13 Member 5 (encoded by SLC13A5), Inducible T Cell Costimulator (encoded by ICOS), Formyl Peptide Receptor 2 (encoded by FPR2), Matrix Metallopeptidase 12 (encoded by MMP12), Tenascin N (encoded by TNN), Asporin (encoded by ASPN), Granzyme A (encoded by GZMA), Osteoglycin (encoded by OGN), Wnt Family Member 2 (encoded by WNT2), Neuropeptide Y (encoded by NPY), Bone Morphogenetic Protein 2 (encoded by BMP-2), Family With Sequence Similarity 169 Member A (encoded by FAM169), Retinol Binding Protein 1 (encoded by RBP1), ISG15 Ubiquitin Like Modifier (encoded by ISG15), Tribbles Pseudokinase 3 (encoded by TRIB3), Ubiquitin Specific Peptidase 18 (encoded by USP18), Claudin 1 (encoded by CLDN1), Myocilin (encoded by MYOC), C-X-C Motif Chemokine Ligand 9 (encoded by CXCL9), Interferon Induced Protein With Tetratricopeptide Repeats 3 (encoded by IFIT3), RRAD, Ras Related Glycolysis Inhibitor And Calcium Channel Regulator (encoded by RRAD) Interferon Alpha Inducible Protein 27 (encoded by IF127). Thus, in some embodiments, the protein of interest is a protein selected from this group. The protein of interest may also be a mammalian orthologue to the above mentioned proteins.

Detailed information on the human genes encoding the proteins of interest in the present invention may be found e.g. in the database GeneCards® (www.genecards.org).

In one embodiment, the protein of interest is a protein involved in tissue repair or re-generation.

In some embodiments, the composition is for use in a method for treatment of a damaged tissue in a subject, said method the composition comprises administering said composition to the site of the damaged tissue.

In some embodiments, the damaged tissue is bone tissue.

The person skilled in the art realizes that the present disclosure is not limited to the preferred embodiments described above, but that modifications and variations are possible within the scope of the appended claims. Additionally, variations to the disclosed embodiments can be understood and effected by the skilled person in practicing the claimed disclosure, from a study of the drawings, the disclosure, and the appended claims.

All references cited herein are expressly incorporated by reference.

The examples below are included to further illustrate the invention and shall not be considered as limiting of the invention.

### EXAMPLES

The examples below are included to illustrate the invention in terms of some embodiments thereof and shall not be construed as limiting the scope of the invention, which is that of the appended claims.

### Example 1

### MnO Sample Preparation.

Commercial pure MnO (Sigma-Aldrich, Sweden, 99.9%) in the form of powder (grain size 1-2 um), were incubated in cell culture medium (DMEM) for 24-72 h, rinsed in saline and distilled water and dry sterilized at 160ºC for 2 h.

The chemical composition of the samples were analysed by ToF-SIMS before and after incubation in DMEM, as described (Nygren et al., 2017).

### Human Mesenchymal Stem Cell Culture.

The human embryonic stem cell (hESC) lines used in this study were SA167MFG-hESC and AS034.1MFG-hESC at passage 12 and 44 respectively derived and characterized in our previous study (Bigdeli et al., 2007). Note that the stem cells adhere to plastic dishes and can be cultured in dishes.

Expansion of hESCs

In this study hESCs were expanded and differentiated toward the osteogenic lineage directly onto tissue culture plastic without any supportive coating. In brief, cells were expanded in conditioned hES medium as described earlier (Bigdeli et al., 2007) containing 80% KnockOut™ DMEM (Gibco-BRL/Invitrogen, Gaithersburg, MD, USA), 20% KnockOut™ serum replacement (SR; Gibco-BRL/Invitrogen), 2 mM L-Glutamine (Gibco-BRL/Invitrogen), 0.1 mM [symbol] -mercaptoethanol (Gibco-BRL/Invitrogen) and 1% NEAA (nonessential amino acids; Gibco-BRL/Invitrogen) on Primaria® dishes (Falcon, surface modified polystyrene non-pyrogenic; Becton Dickinson, Franklin Lakes, USA) and were incubated in a humidified atmosphere at 37° C. and 5% CO₂ (Heraeus BBD6220). The SA167MFG-hESC and AS034.1MFG-hESC were passaged every 4 to 6 days and the medium was changed every second day.

### Exposure of Stem Cells to Metal Oxide/CHA

Undifferentiated hESCs were cultured on regular tissue culture plastic without pre-differentiation stages such as embryoid body (EB) formation.

Cell exposure was performed by adding the CHA-coated metal oxides in different concentrations into the culture medium for 24 hours.

### Von Kossa Staining

Calcium precipites (mainly hydroxyapatite) was studied using von Kossa staining performed by washing the cells in PBS followed by fixation in Glutaraldehyde solution (25% in H₂O Sigma-Aldrich diluted 1:10) for 2 hours. A solution of AgNO₃ (2% w/v: Sigma-Aldrich) was added and the plates were kept in dark for 10 min. The plates were then rinsed three times with distilled H₂O before being exposed to bright light for 15 min. After washing with distilled H₂O, samples were quickly dehydrated adding 100% EtOH prior to microscopic inspection.

### Cell Viability.

hMSCs were seeded onto a 24 well plate at density of 10 000 cells/well. Cells were incubated in growth medium with or without the presence of metal oxides for 24 hours to allow for attachment. Attached cells were considered viable and floating cells non-viable.

### ToF-SIMS.

ToF-SIMS analysis was performed with a TOF.SIMS 5 instrument (ION-TOF GmbH, Münster, Germany) using a Bi3+ cluster ion gun as the primary ion source. Multiple (n=5) regions ranging from 60 μm×60 μm to 105 μm×105 μm were analyzed with a pulsed primary ion beam (Bi3+, 0.24 pA at 25 keV, Dose density 1.12×1011) with a focus of approximately 2 μm and a mass resolution of M/ΔM=5×103 fwhm at m/z 500. All spectra were acquired and processed with the Surface Lab software (version 6.3, ION-TOF GmbH, Münster, Germany) and the ion intensities used for calculations were normalized to the total ion dose of each measurement. ToF-SIMS analysis is surface sensitive and detects atoms and molecules in the first nanometer at the surface. ToF-SIMS is not considered a quantitative analysis.

### Statistical Analysis.

Statistical analysis was performed using t-test. The limit of statistical significance was set at p>0.05.

### Results

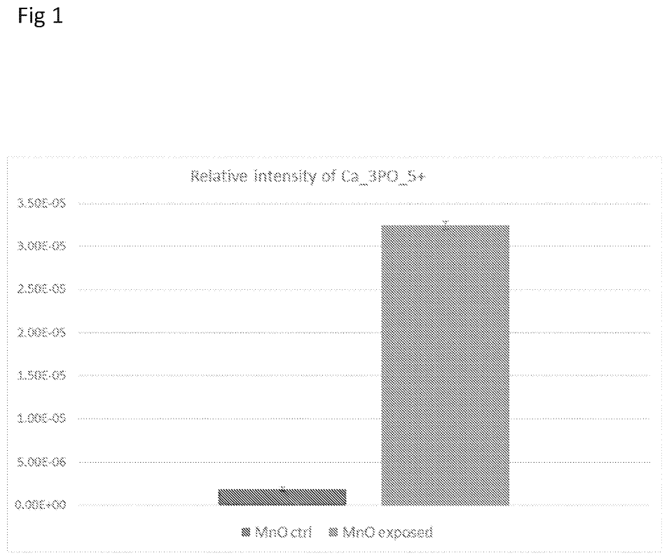

Incubation of MnO in DMEM results in formation of hydroxyapatite, whereas essentially no hydroxyapatite is formed by incubation of MnO in saline control, as shown in FIG. 1. The diagram in FIG. 1 shows the results of a tof-sims analys of an ion (Ca₃PO₅⁺) released from Hydroxyapatite during bombardment in the instrument. This ion is formed during analysis and reveals the presence of hydroxyapatite in the MnO sample incubated in cell culture medium, but not in saline.

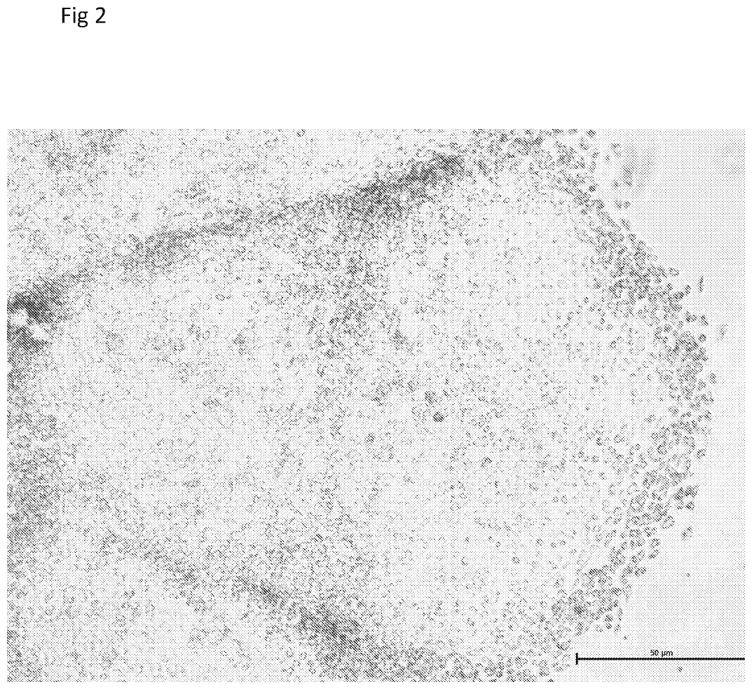

The results of exposing hESCs to MnO, that have been preincubated in MEM for 24-72 h are shown in FIG. 2. The hESCs, adhered to petri dishes and exposed to 0.5 mg/ml of preincubated MnO, showed >90% coverage of cells adhering in colonies to the dishes. Staining the fixed cultures with von Kossa staining showed that the cells contained and were surrounded by HA after exposure to this concentration of MnO preincubated with DMEM. The cell reaction may either be a specific activation of the cells or a stress reaction at exposure to MnO. The pH of the growth medium was stable after exposure to MnO, preincubated with DMEM.

### Example 2

Preparation of HA-RNA Bound to Metal Oxides Through Incubation with Cell Culture Medium (DMEM)

Metal oxides, MnO and MgO (powder), were sterilized by heating in an oven for 2 h at 160° C. The metal oxides were then incubated in Falcon tubes with 6 ml of:
a. sterile DMEM containing 10% sterile fetal calf serum and 10 μg of RNA, or in
b. sterile saline containing 10 μg of RNA as control

After 6 h, the oxides were spun down at 1300 rpm for 3 minutes. Pellets were rinsed with sterile water 3 times. The pellets were dried at 60° C. over night and then analysed by ToF-SIMS.

Samples were analyzed with ToF-SIMS in positive and negative polarity. ToF-SIMS analysis was performed on the powders samples using a TOF.SIMS 5 instrument (ION-TOF GmbH, Münster, Germany). Using a 25 keV Bi cluster ion gun as the primary ion source. The samples were analyzed using a pulsed primary ion beam (Bi₃⁺⁺, 0.34 pA at 50 keV) with a focus of approximately 2 μm using the high current bunched mode(Sodhi, 2004) to obtain high mass resolution spectra. The mass resolution using this setup was at least M/ΔM=5000 fwhm at m/z 500. All spectra were acquired and processed with the Surface Lab software (version 6.4, ION-TOF GmbH, Munster, Germany). The spectra were internally calibrated to signals of [C]⁺, [CH₂]⁺, [CH]⁺, [C₅H₁₅ PN₄]⁺ and [C₂₇H₄₅]⁺ for the positive ion mode and [C]⁻, [CH₂]⁻, [C₂]⁻, [C₃]⁻ for the negative ion mode.

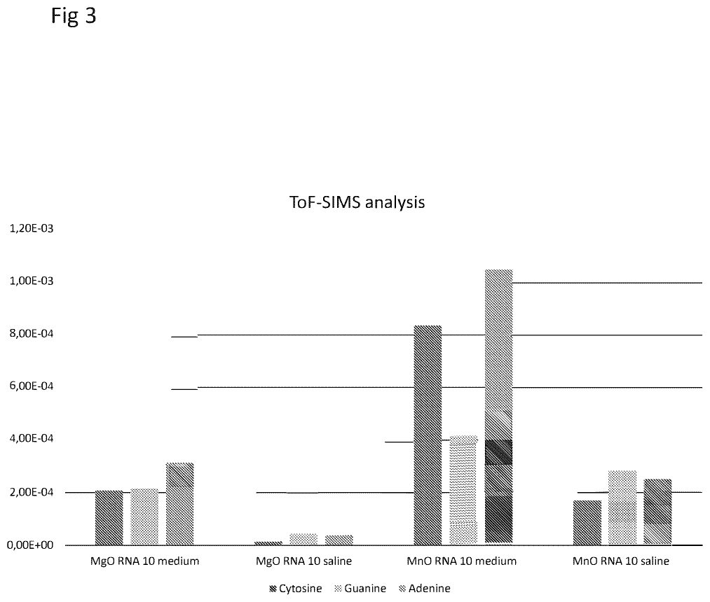

Peaks specific for Cytosine, Guanine and Adenine were analyzed. An average ion signal from each peak was collected and normalized to the total ion count for each sample. Results are shown in FIG. 3.

Adenine, Cytosine and Guanin were detected on the metal oxides. The levels of adsorbed bases is higher in samples incubated with cell culture medium, containing Ca²⁺ ions. This indicates that RNA and Ca²⁺ forms hydroxyapatite on the surface of the metal oxide particles.

### Example 3

### Effect of Type of Hydrogel on the Binding of RNA to Metal Oxide Particles

1. Three types of DSMs, termed A, B, and C below, were used in the experiment.

| | d (0.5) | Continuous | | | |
|---|---|---|---|---|---|
| DSM | μm | phase | Emulsifier | Crosslinker | pH |
| A | 60 μm | Non-polar | SPAN80 | Phosphate- | Basic |
| | | solvent | | based | |
| B | 41 μm | Non-polar | SPAN80 | Phosphate- | Acidic |
| | | solvent | | based | |
| C | 41 μm | Non-polar | Rhodafac | Oxirane- | Acidic |
| | | solvent | PA17 | derivative | |

“d(0.5)” indicates that 50% of the microspheres have a diameter less than the indicated value

All three DSMs were autoclaved as a dry powder prior to experiment (all 3 dry powders are stable at 121° ° C. for 20 min in sealed vial).
2. MnO and MgO were sterilized by heating in oven for 2 h at 160° C.
3. DMEM medium, fetal bovine serum, water for injection, Falcon tubes were sterile.
4. RNA solution at 1 mg/ml (in sterile saline) was prepared on the day of experiment and used directly.
5. On the day of experiment, reactions containing following components were prepared:
   a. 0.3 g DSM (powder)+6 ml DMEM medium with serum+200 μl MnO+10 μg RNA (10 μl)
   b. 0.3 g DSM (powder)+6 ml DMEM medium with serum+200 μl MgO+10 μg RNA (10 μl)

In total 6 reactions were prepared (2 tubes per each DSM).

Final concentration of DSM in the above mixtures is 5%. The resulting gels are denoted “A”, “B”, and “C” corresponding to the DSM used in preparation of the gel,

Each DSM was swelled in DMEM medium overnight and then other components were added and complete mix was incubated with gentle rotation (at RT) for 6 h.

After 6 h, the oxides+gel were spun down (approx. 1300 rpm for 3 minutes). Pellet was rinsed with water (5-6 ml). Spinning/washing step was repeated 3 times.

Gel+oxide pellets were dried overnight at 50-60° C. Dried samples are sent for analysis by Tof-SIMS.

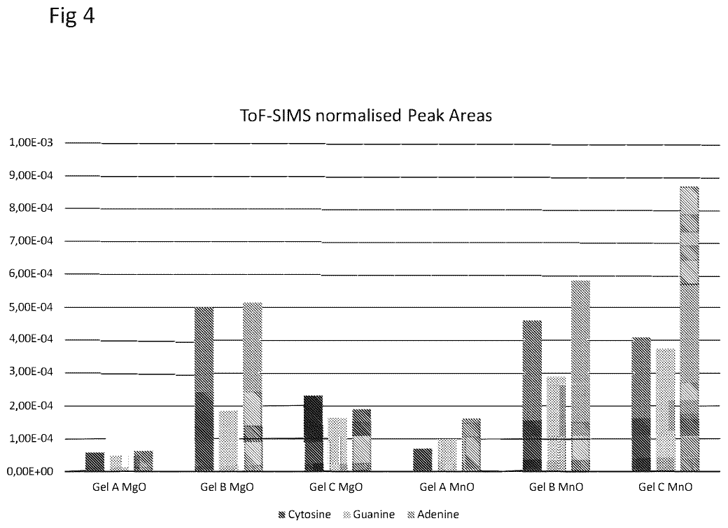

The results (FIG. 4) show that the gel composition has a major effect on the binding of RNA to MgO and MnO. The binding of RNA in gel DSM 480 is 10 times higher than in the gel DSM 52. The differences in chemical composition between these gels is the acidity. The Gel B is cross-linked by phosphate and has acidic properties, whereas Gel A is cross-linked by phosphate and has basic properties. The Gel C is cross-linked by epichlorhydrine and has acid properties. The results suggest that the gel matrix should be acidic in order to support the binding of RNA to metal oxides, most probably by the formation of a layer of hydroxyapatite on the surface of the metal oxides in the presence of Ca²⁻- ions

### Example 4

This example illustrates a protocol for assessing effective mRNA delivery using a composition according to one embodiment of the invention.

Cross-linked starch (Sterile 0.05 g) is swollen to form a gel in 1 ml of sterile saline, also containing 0.5 mg of MnO and 200 μg of mRNA encoding a protein of interest. A portion of the gel (100 μl) is injected into rat tibia bone with a drilled rift as described previously (Nygren et al., 2017), and the bone is allowed to heal for 24-96 hours. The rat is euthanized and the bone is dissected out. Preparations of histological sections is made and the protein of interest encoded for by the injected mRNA is detected by immunohistochemistry (Uhlen et al., 2015).

### REFERENCES

Atyabi, et al. (2006). Cross-linked starch microspheres: effect of cross-linking condition on the microsphere characteristics. Arch Pharm Res, 29(12), 1179-1186.
Aulton, E. M. (2002). Pharmaceutics: The Science of Dosage Form Design (2nd ed.). Edinburgh: Churchill Livingstone.
Banker et al. (2002). Modern Pharmaceutics (4th ed.). Boca Raton: CRC Press.
Bigdeli et al. (2007, Jan). Adaptation of human embryonic stem cells to feeder-free and matrix-free culture conditions directly on plastic surfaces. J Biotechnol., 133(1), 146-153.
Fadrosh et al. (2011, Sep. 29). Separation of Single-stranded DNA, Double-stranded DNA and RNA from an Environmental Viral Community Using Hydroxyapatite Chromatography. Journal of Visualized Experiments, 55(e3146).
Fang, et al. (2008). Preparation of crosslinked starch microspheres and their drug loading and releasing properties. Carbohydrate Polymers, 74(3), 379-384.
Fraceto, L. F., & Ribeiro de Araújo, D. (Eds.). (2015). Microspheres, Technologies, applications and role in drug delivery systems. New York, U.S.: Nova Science Publishers, Inc.
Lachman et al. (1986). The Theory and Practice of Industrial Pharmacy (3rd ed.). Philadelphia: Lea & Febiger.
Liu et al. (2020). Facile synthesis of biodegradable flower-like hydroxyapatite for drug and gene delivery. Journal of Colloid and Interface Science, 570, 402-410.
Ludwig et al. (2021, Oct. 13). European Multicenter Study on Degradable Starch Microsphere TACE: The Digestible Way to Conquer HCC in Patients with High Tumor Burden. cancers, 13, 5122.
Malone et al. (1989). Cationic liposome-mediated RNA transfection. Proc Natl Acad Sci USA, 86(16), 6077-6081.
Nygren et al. (2017, June). Mg-corrosion, hydroxyapatite and bone healing. Biointerphases, 12(2).
Schlake et al. (2012). Developing mRNA-vaccine technologies. RNA Biol., 9(11), 1319-1330.
Swarbrick, J. (2006). Encyclopedia of Pharmaceutical Technology (4th ed.). Boca Raton: CRC Press.
Uhlén et al. (2015). Tissue-based map of the human proteome. Science, 347(6220), 1260419.
Zantye et al. (2021). Design of a Biocompatible Hydroxyapatite-Based Nanovehicle for Efficient Delivery of Small Interference Ribonucleic Acid into Mouse Embryonic Stem Cells. Mol. Pharmaceutics, 18, 796-806.

1. A pharmaceutically acceptable aqueous gel composition comprising a gelling agent, magnesium and/or manganese, or bivalent ions thereof, and an mRNA molecule encoding a protein of interest.

2. The composition according to claim 1, comprising manganese, or bivalent ions thereof.

3. The composition according to claim 1, wherein the magnesium and/or manganese, or bivalent ions thereof, is present in an amount sufficient to induce hydroxyapatite formation under physiological conditions.

4. The composition according to claim 1, wherein the gelling agent is selected from starch or derivatives thereof; agar/agarose or derivatives of agar/agarose; hyaluronic acid or derivatives of hyaluronic acid; chitosan; gelatin; and dextran.

5. The composition according to claim 1, wherein the protein of interest is a protein involved in tissue repair, tissue re-generation, or tissue generation.

6. The composition according to claim 1, wherein the protein is selected from the group consisting of Phosphate-regulating neutral endopeptidase, X-linked (encoded by PHEX), heat shock protein 90-alpha (encoded by HSP90AA1), Chordin Like 2 (encoded by CHRDL2), short transient receptor potential channel 4 (encoded by TRPC4), pannexin 3 (encoded by PANX3), Collagen Type XXIV Alpha 1 (encoded by COL24A1), the gene product of ATP283, Pleckstrin Homology Domain Containing B1 (encoded by PLEKHB1), Leukocyte immunoglobulin-like receptor subfamily B member 4 (encoded by LILRB4), Anoctamin 5 (encoded by ANO5), ChaC Glutathione Specific Gamma-Glutamylcyclotransferase 1 (encoded by CHAC1), Dynein Axonemal Intermediate Chain 2 (encoded by DNAI2), Aggrecan (encoded by ACAN), Integrin Subunit Alpha 10 (encoded by ITGA10), Fibromodulin (encoded by FMOD), Secreted Phosphoprotein 1 (a.k.a. osteopontin, encoded by SPP1), Apolipoprotein C1 (encoded by APOC1), Patched 2 (encoded by PTCH2), Apolipoprotein E (encoded by APOE), Triggering Receptor Expressed On Myeloid Cells 2 (encoded by TREM2), podoplanin (encoded by PDPN), Solute Carrier Family 13 Member 5 (encoded by SLC13A5), Inducible T Cell Costimulator (encoded by ICOS), Formyl Peptide Receptor 2 (encoded by FPR2), Matrix Metallopeptidase 12 (encoded by MMP12), Tenascin N (encoded by TNN), Asporin (encoded by ASPN), Granzyme A (encoded by GZMA), Osteoglycin (encoded by OGN), Wnt Family Member 2 (encoded by WNT2), Neuropeptide Y (encoded by NPY), Bone Morphogenetic Protein 2 (encoded by BMP-2), Family With Sequence Similarity 169 Member A (encoded by FAM169), Retinol Binding Protein 1 (encoded by RBP1), ISG15 Ubiquitin Like Modifier (encoded by ISG15), Tribbles Pseudokinase 3 (encoded by TRIB3), Ubiquitin Specific Peptidase 18 (encoded by USP18), Claudin 1 (encoded by CLDN1), Myocilin (encoded by MYOC), C-X-C Motif Chemokine Ligand 9 (encoded by CXCL9), Interferon Induced Protein With Tetratricopeptide Repeats 3 (encoded by IFIT3), RRAD, Ras Related Glycolysis Inhibitor And Calcium Channel Regulator (encoded by RRAD), and Interferon Alpha Inducible Protein 27 (encoded by IFI27).

7. A method for inducing or facilitating repair, re-generation or generation of tissue in a human or animal subject, said method comprising administering the composition according to claim 1 to the site of the tissue to be repaired, re-generated or generated.

8. The method according to claim 7, wherein the method for inducing or facilitating repair, re-generation or generation of tissue is for facilitating or improving healing of tissue damaged due to trauma and/or disease.

9. The method according to claim 7, wherein the tissue is bone tissue.

10. The method according to claim 9, wherein the method for inducing or facilitating repair, re-generation or generation of tissue is performed in in connection with spinal fusion, or implantation of implants in bone tissue, such as prosthetic joints and dental implants.
